(19) United States
(12) Patent Application Publication
(10) Pub. No.: US 2015/0250734 A1
(43) Pub. Date: Sep. 10, 2015

(54) Stable pharmaceutical compositions of saxagliptin or salts thereof

(71) Applicant: WOCKHARDT LIMITED, Aurangabad (IN)

(72) Inventors: Girish Kumar Jain, Delhi 4, (IN); Venkataramana Naidu, Andhra Pradesh, (IN); Balasaheb Parshuram Wagh, Maharashtra (IN); Vijay Suggala, Andhra Pradesh, (IN)

(21) Appl. No.: 14/436,881

(22) PCT Filed: May 3, 2013
(86) PCT No.: PCT/IB2013/053526

§ 371 Date: Apr. 18, 2015

(30) Foreign Application Priority Data
Dec. 21, 2012 (IN) 3582/MUM/2012
Dec. 21, 2012 (IN) 3583/MUM2012

(51) Int. Cl. A61K 9/28; A61K 9/20; A61K 31/403

(52) U.S. Cl. CPC A61K 9/2886; A61K 9/2866; A61K 9/284; A61K 9/2853; A61K 9/288; A61K 9/2813; A61K 9/282; A61K 9/2826; A61K 31/403; A61K 9/2086; A61K 9/2893

(57) ABSTRACT

The present invention refers to a stable pharmaceutical composition of saxagliptin or salts thereof. In particular, the invention relates to stable comprises a core and two or more layers coated on the core, wherein the composition or the inner first coat is free of polyvinyl alcohol. Such composition of saxagliptin may exhibit relatively improved storage stability and particularly, levels of degradants in the formulation during storage can be effectively controlled. The invention also includes a process of preparing such compositions and method of treating type-II diabetes mellitus by administering the composition to a patient in need thereof.

### FIELD OF THE INVENTION

The present invention relates to a stable pharmaceutical composition of saxagliptin or salts thereof. In particular, the invention relates to stable comprises a core and two or more layers coated on the core, wherein the composition or the inner layer/coat is free of polyvinyl alcohol. Such composition of saxagliptin may exhibit relatively improved storage stability and particularly, levels of degradants in the formulation during storage can be effectively controlled. The invention also includes a process of preparing such compositions and method of treating type-II diabetes mellitus by administering the composition to a patient in need thereof.

### BACKGROUND OF THE INVENTION

Saxagliptin is an orally active inhibitor of the dipeptidyl peptidase-4 (DPP4) enzyme. After a meal intake, insulinotropic hormone glucagon-like peptide-1 (GLP-1) is released which in turn induces insulin release from the pancreas. Some of the GLP-1 is inactivated by the DPP4 present in plasma and intestinal capillary endothelium. Therefore, if the DPP4 is inhibited, more GLP-1 will be available to activate insulin release from the pancreas. The advantage of this mechanism of insulin release is that insulin is secreted only in response to a meal. Therefore, problems of hypoglycemia associated with other diabetes drugs are less likely with a DPP4 inhibitor.

Chemically saxagliptin is (1 S,3S,5S)-2-[(2S)-2-Amino-2-(3-hydroxytricyclo[3.3.1.1]dec-1-yl)acetyl]-2-azabicyclo[3.1.0]hexane-3-carbonitrile,monohydrate or (1 S,3S,5S)-2-[(2S)-2-Amino-2-(3-hydroxyadamantan-1-yl)acetyl]-2-azabicyclo[3.1.0]hexane-3-carbonitrile with the structure

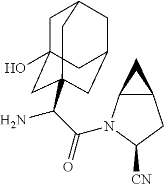 [SMILES: N#C[C@@H]1C[C@@H]2C[C@@H]2N1C(=O)[C@@H](N)C12CC3CC(CC(O)(C3)C1)C2]

Saxagliptin alone or in combination with other anti-diabetic agents are indicated as an adjunct to diet and exercise to improve glycemic control in adults with type-II diabetes mellitus in multiple clinical conditions. It is marketed in the United States in the form of tablets under the brand name Onglyza®.

It is well known in the art that saxagliptin is an unstable compound and it is prone to an intra-molecular cyclization. The resultant degradant, cyclic amidine (mainly cis-cyclic amidine) is not therapeutically active and therefore, its formation is not desirable. This cyclization reaction can occur both in solid state and solution state. The rate of intra-molecular cyclization of saxagliptin is accelerated when formulations are subject to commonly used processing activities such as wet granulation, roller compaction, or tabletting. In addition, most commonly used excipients, when mixed with saxagliptin, can accelerate the rate of cyclization. Moreover, the level of cis-cyclic amidine increases when the drug to excipient ratio increases posing more challenges for low strength dosage forms. Thus, these properties of saxagliptin posses major challenge in devising conventional and stable dosage form with ease of manufacture.

Several studies have been conducted to address the formulation and drug release systems of DPP4 inhibitor's and attempts have also been made to improve the formulation stability.

U.S. Pat. No. 6,395,767 discloses a DPP4 inhibiting compound, saxagliptin and its use in treating type-II diabetes mellitus.

PCT Publication No. WO 2011/052825 discloses a composition of DPP4 inhibitors and anti-diabetic compounds for use in the treatment of diabetes.

U.S. Patent Application Publication No. 2005/0208133 discloses a multiple drug release systems, its composition and methods of its preparation.

PCT Publication No. WO 2002/085335 discloses a composition providing control release of medicament. The composition contains several coatings, which controls the release of the medicament.

U.S. Pat. No. 7,951,400 discloses a saxagliptin tablet containing several coating layers of polyvinyl alcohol.

Although various attempts have been made earlier for improving the stability of saxagliptin during the process of formulation and storage, the compositions disclosed in the prior art either suggests incorporating the drug in the core of the formulation or applying specialized polymer coatings when drug is placed in coating.

Still, there exists an enduring need for alternative, improved and stable pharmaceutical composition of saxagliptin, which exhibits excellent storage stability and that to without placing saxagliptin in the core.

### SUMMARY OF THE INVENTION

In one aspect, the present invention provides a stable pharmaceutical composition of saxagliptin or salts, hydrates thereof comprising:

(a) at least one core;
(b) at least one first coating layer coated over the core comprising one or more pharmaceutical excipients and optionally with one or more polymers;
(c) at least one second coating layer disposed over the first coating layer comprising saxagliptin or salts, hydrates thereof, one or more pharmaceutical excipients and optionally with one or more polymers; and
(d) optionally, an outer coating layer disposed over the second coating layer comprising one or more pharmaceutical excipients and optionally with one or more polymers, wherein said core does not contain saxagliptin, or salts, hydrates thereof and the composition is free of polyvinyl alcohol.

In another aspect, the present invention provides a stable pharmaceutical composition of saxagliptin or salts, hydrates thereof comprising:

(a) at least one core;
(b) at least one first coating layer coated over the core optionally comprising one or more polymers;
(c) at least one second coating layer disposed over the first coating layer comprising saxagliptin or salts, hydrated thereof and one or more polymers; and
(d) optionally, an outer coating layer disposed over the second coating layer comprising one or more polymers,
wherein said core does not contain saxagliptin, or salts, hydrates thereof and said first coating layer is free of polyvinyl alcohol.

In another general aspect, the polymer in the second coating layer and the outer coating layer is different that the polymer in the first coating layer.

In another aspect, the present invention provides a tablet comprising:

(a) a core comprising one or more pharmaceutical excipients, and optionally one or more anti-diabetic agents;
(b) at least one first coating layer coated over the core comprising hydroxypropyl methylcellulose;
(c) at least one second coating layer disposed over the first coating layer comprising saxagliptin or salts, hydrated thereof, one or more pharmaceutical excipients and optionally with one or more polymers; and
(d) optionally, an outer coating layer disposed over the second coating layer comprising one or more pharmaceutical excipients and optionally with one or more polymers,
wherein said core does not contain saxagliptin, or salts, hydrates thereof and the composition is free of polyvinyl alcohol.

In another aspect, the present invention provides a tablet comprising:

(a) a core comprising one or more pharmaceutical excipients, and optionally one or more anti-diabetic agents;
(b) at least one first coating layer coated over the core comprising hydroxypropyl methylcellulose;
(c) at least one second coating layer disposed over the first coating layer comprising saxagliptin or salts, hydrated thereof, one or more pharmaceutical excipients and optionally with one or more polymers; and
(d) optionally, an outer coating layer disposed over the second coating layer comprising one or more pharmaceutical excipients and optionally with one or more polymers,
wherein said core does not contain saxagliptin, or salts, hydrates thereof and said first coating layer is free of polyvinyl alcohol.

In another aspect, the present invention provides a tablet comprising:

(a) a core comprising one or more pharmaceutical excipients, and optionally one or more anti-diabetic agents;
(b) at least one first coating layer coated over the core comprising hydroxypropyl methylcellulose;
(c) at least one second coating layer disposed over the first coating layer comprising saxagliptin or salts, hydrated thereof and polyvinyl alcohol; and
(d) optionally, an outer coating layer disposed over the second coating layer comprising polyvinyl alcohol,
wherein said core does not contain saxagliptin, or salts, hydrates thereof and said first coating layer is free of polyvinyl alcohol.

In another general aspect, the composition further comprises an outermost layer containing colorants and one or more polymers to differentiate compositions of various strengths. The components of the outermost layer may be similar as in the outer layer.

In another aspect, the present invention provides a process for preparation of a stable pharmaceutical composition comprising saxagliptin or salts, hydrates thereof, which process comprises of:

(a) providing at least one core;
(b) coating the core with at least one first coating layer comprising one or more pharmaceutical excipients and optionally with one or more polymers;
(c) coating at least one second coating layer comprising saxagliptin or salts, hydrated thereof, one or more pharmaceutical excipients and optionally, one or more polymers over the first coating layer;
(d) optionally, coating an outer coating layer comprising one or more pharmaceutical excipients and optionally one or more polymers, over the second coating layer; and
(e) optionally, coating the outermost protective coating layer comprising one or more polymers and colorant over the outer coating layer,
wherein said core does not contain saxagliptin, or salts, hydrates thereof and the composition is free of polyvinyl alcohol.

In another general aspect, the coating layers on the core are applied by spray coating. Perforated pan coaters and fluid bed coaters can be used for the coating.

In another general aspect, in the process for preparation of stable pharmaceutical composition comprising saxagliptin or salts, hydrates thereof, the first coating layer, the second coating layer, the outer coating layer, and optionally outermost protective coating layer each are applied as a suspension of the polymer in a coating solvent.

In another general aspect, the stable pharmaceutical composition of saxagliptin or salts, hydrates thereof retains at least 90% w/w of total potency of saxagliptin after storage at 40° C. and 75% relative humidity for at least 3 months.

In another aspect, the present invention provides a method of treating type-II diabetes mellitus in a patient which method comprising administering the pharmaceutical composition as substantially described herein to the patient.

Embodiments of the pharmaceutical composition may include one or more of the following features. For example, the pharmaceutically acceptable excipients may include diluents, disintegrants, binders, bulking agents, anti-adherents, anti-oxidants, buffering agents, colorants, flavoring agents, coating agents, plasticizers, stabilizers, preservatives, lubricants, glidants, chelating agents, and the like known to the art used either alone or in combination thereof.

### DETAILED DESCRIPTION OF THE INVENTION

The inventors of the present invention have surprisingly found that it is possible to formulate a stable pharmaceutical composition of saxagliptin, which can exhibit superior chemical and physical stability without using polyvinyl alcohol polymer in the composition or in a specific coating layer.

The stable pharmaceutical composition of the present invention comprises saxagliptin, or salts, hydrates thereof. The composition comprises one or more cores and two or more layers coated on the core, wherein the composition or the first coat is free of polyvinyl alcohol. The core in the composition does not contain saxagliptin, or salts, hydrates thereof.

The pharmaceutical composition of the present invention exhibits excellent storage stability over the storage period.

In an embodiment, the stable pharmaceutical composition of the present invention may be characterized by lower level of degradant cis-cyclic amidine.

In another embodiment, the stable pharmaceutical composition of saxagliptin or salts, hydrates thereof retains at least 90% w/w of the total potency of saxagliptin after storage at 30° C. and 60% relative humidity, at 40° C. and 75% relative humidity, or at

° C. and 60% relative humidity for at least 3 months.

The term “saxagliptin” used throughout the specification refers to not only saxagliptin per se, but also various pharmaceutically acceptable salts and pharmaceutically acceptable hydrates thereof.

The term “core” used throughout the specification refers to a “core”, “tablet core”, “placebo”, “placebo core tablet”, “tablet core composition” or “core composition”.

The core employed in the composition of the invention may include conventional pharmaceutical excipients to enable formation of a pharmaceutically acceptable solid core. The core may be in the form of a tablet, bead, beadlet, or pill.

In an embodiment, the core may contain one or more anti-diabetic agents, other than saxagliptin, in an amount within the range from about 0.1 to about 70% wt/wt and preferably from about 1 to about 50% w/w of the composition.

The core may be formed of one or more pharmaceutical excipients selected from, but not limited to one or more of bulking agents or fillers, binders, disintegrants, and lubricants.

In an embodiment, the core preferably contain a) at least one bulking agent or filler; b) optionally at least one binder; c) optionally at least one disintegrant; and d) preferably but optionally at least one lubricant, wherein a) the bulking agent or filler is present in an amount within the range from about 1 to about 95% w/w, preferably from about 10 to about 85% w/w; b) the binder is present in an amount within the range from about 0 to about 20% w/w, preferably from about 1 to about 10% w/w; c) the disintegrant is present in an amount within the range from about 0 to about 20% w/w, and preferably from about 0.25 to about 10% w/w; and d) the lubricant is present in an amount within the range from about 0 to about 5% w/w, preferably from about 0.2 to about 2% w/w of the composition.

In another embodiment, the bulking agents are microcrystalline cellulose and lactose monohydrate; the disintegrant is croscarmellose sodium; and the lubricant is magnesium stearate.

The cores present in the composition of the invention can be prepared by a variety of processes and order of addition of excipients. The utility of these formulations is not limited to a specific dosage form or manufacturing process. The cores may be manufactured by wet granulation, dry granulation, direct blending or any other pharmaceutically acceptable process.

In an embodiment, the process of preparing the cores includes the steps of blending the one or more excipients such as bulking agent, optionally binder and optionally disintegrant. A lubricant will be preferably added to the blend to facilitate tablet formation.

The bulking agents or fillers may be present in the core in an amount within the range from about 1 to about 95% w/w and preferably from about 10 to about 85% w/w of the composition. Examples of bulking agents or fillers suitable for use herein include, but are not limited to, cellulose derivatives such as microcrystalline cellulose or wood cellulose, lactose, sucrose, starch, pregelatinized starch, dextrose, mannitol, fructose, xylitol, sorbitol, corn starch, modified corn starch, inorganic salts such as calcium carbonate, calcium phosphate, dicalcium phosphate, calcium sulfate, dextrin/dextrates, maltodextrin, compressible sugars, and other known bulking agents or fillers, and/or mixtures thereof, preferably microcrystalline cellulose.

The binder may be present in the core in an amount within the range from about 0 to about 20% w/w, preferably from about 1 to about 10% w/w of the composition. Examples of binders suitable for use herein include, but are not limited to, hydroxypropyl cellulose, corn starch, pregelatinized starch, modified corn starch, polyvinyl pyrrolidone (PVP) (molecular weight ranging from about 5,000 to about 1,000,000, preferably about 40,000), hydroxypropyl methylcellulose (HPMC), lactose, gum acacia, ethyl cellulose, cellulose acetate, as well as a wax binder such as carnauba wax, paraffin, spermaceti, polyethylenes or microcrystalline wax, as well as other conventional binding agent and/or mixtures thereof, preferably hydroxypropyl cellulose.

The disintegrant may be present in the core in an amount within the range from about 0 to about 20% w/w, preferably from about 0.25 to about 10% w/w of the composition. Examples of disintegrants suitable for use herein include, but are not limited to, croscarmellose sodium, crospovidone, starch, potato starch, pregelatinized starch, corn starch, sodium starch glycolate, microcrystalline cellulose, low substituted hydroxypropyl cellulose or other known disintegrant, preferably croscarmellose sodium. The lubricant may be present in the core in an amount within the range from about 0.1 to about 5% w/w, preferably from about 0.2 to about 2% w/w of the composition. Examples of tableting lubricants suitable for use herein include, but are not limited to, magnesium stearate, zinc stearate, calcium stearate, talc, carnauba wax, stearic acid, palmitic acid, sodium stearyl fumarate or hydrogenated vegetable oils and fats, or mixtures thereof, preferably magnesium stearate.

The first coating layer of the composition comprises one or more pharmaceutical excipients and optionally one or more polymers.

The amount of polymer in the first coating layer is more than 50% by weight of polymer, preferably more than 80% by weight of polymer, and most preferably more than 90% by weight of polymer relative to the total weight of the first coating layer.

The coating formulation for first coating contains at least one polymer and a coating solvent, which preferably is water, which is used for processing and removed by drying. Suitable polymer for first coating layer may be selected from, but not limited water-soluble polymer, water-insoluble polymer, or mixtures thereof. Particularly, water-soluble polymers are preferred.

Examples of polymers suitable for first coating layer include, but not limited to hydroxypropyl methylcellulose, ethyl cellulose, methacrylic polymers or hydroxypropyl cellulose, preferably hydroxypropyl methylcellulose. The coating layer may also optionally include a plasticizer such as triacetin, diethyl phthalate, tributyl sebacate or polyethylene glycol (PEG), preferably PEG; and an anti-adherent or glidant such as talc, fumed silica or magnesium stearate, opacifying agent such as titanium dioxide. The coating layer may also include iron oxide based colorants.

Examples of suitable coating solvents includes, but not limited to water, ethanol, methanol, and isopropyl alcohol, with water being preferred.

The first coating layer which is free of polyvinyl alcohol will preferably be formed by coating polymer layer in an amount within the range from about 10 to about 99%, preferably from about 20 to about 99% w/w of the first coating layer, optionally plasticizer in an amount within the range from about 1 to about 30%, preferably from about 5 to about 20% w/w of the first coating layer, and anti-adherent or glidant in an amount within the range for about 15 to about 30%, preferably from about 10 to about 15% w/w of the first coating layer.

The first coating layer may be present in an amount within the range from about 1 to about 5%, preferably from about 1 to about 3% w/w of the composition.

The second coating layer of the composition of the invention comprises saxagliptin, or salts, hydrates thereof, one or more pharmaceutical excipients and optionally, one or more polymers. Suitable coating solvent is employed to facilitate the coating, which preferably is water, which is used for processing and removed by drying.

The amount of saxagliptin, or salts, hydrates thereof in the second coating layer is in the range from about 0.25 to about 70%, preferably from about 20 to about 50% w/w, based on the weight of the second coating layer.

The amount of polymer in the second coating layer may range from about 30 to about 99.5%, preferably from about 40 to about 60% w/w of the second coating layer.

The second coating layer (containing saxagliptin) may be present in an amount within the range from about 0.25 to about 70%, preferably from about 1 to about 50% w/w of the composition.

The outer coating layer of the composition of the invention comprises one or more pharmaceutical excipients and optionally, one or more.

In an embodiment, the composition of the invention includes an outer layer where the coating suspension is prepared as in the case of the second coating suspension but without saxagliptin. The coating suspension is then can be coated onto the previously coated composition as described for the first coating and second coating to form a protective coating layer thereon.

The outer protective coating layer will preferably be similar in composition to the second coating layer except without containing saxagliptin.

The composition of the present invention further may comprise an outermost protective layer comprising one or more polymers and one or more pharmaceutical excipients.

The outermost coating layer where present will preferably be similar in composition to the outer protective coating layer and will include colorant as desired, such as within the range from about 0.5 to about 5.0% w/w, based on the total weight of the outermost coating layer.

The outer and outermost protective coating layer if present, may each be present in an amount within the range from about 1 to about 10%, preferably from about 1 to about 5% w/w of the composition.

Pharmaceutical excipients suitable for employing in the coating layers of the composition may include, nut not limited to, bulking agents or diluents, binders, plasticizers, lubricants, colorants, pH adjusting agents, or mixtures thereof.

The invention further provides a process for preparation of stable pharmaceutical composition of saxagliptin or salts, hydrates thereof, the process comprises of:

(a) providing at least one core;
(b) coating the core with at least one first coating layer comprising one or more pharmaceutical excipients and optionally, one or more polymers;
(c) drying the coated core to form first coating thereon;
(d) coating at least one second coating layer comprising saxagliptin or salts, hydrates thereof, one or more pharmaceutical excipients and optionally, one or more polymers over the first coating layer;
(d) drying the coated core to form second coating thereon;
(e) optionally, coating an outer coating layer comprising one or more pharmaceutical excipients and optionally, one or more polymers over the second coating layer;
(e) optionally, drying the coated core to form outer coating thereon;
(f) optionally, coating the outermost protective coating layer comprising one or more coating polymer and colorant over the outer coating layer; and
(g) optionally, drying the coated core to form outermost coating thereon.

In a further embodiment, in preparing the coated composition of the invention, coating suspensions which include coating polymer in water are prepared. Other coating solvents which may be employed include ethanol, methanol, and isopropyl alcohol, with water being preferred. Composition, which is placebo (contain no medicament) and formed cores are coated with the first coating suspension and are dried. The second coating layer suspension containing medicament and coating polymer is applied over the so-coated cores, which are then dried.

The cores present in the composition of this invention can be prepared by a variety of processes and order of addition of excipients. The utility of these formulations is not limited to a specific dosage form or manufacturing process. The cores may be manufactured by wet granulation, dry granulation, direct blending or any other pharmaceutically acceptable process.

In accordance with the present invention, a preferred method is provided for preparing the cores employed in the composition of the invention which includes the steps of blending the one or more excipients such as bulking agent, optionally binder and optionally disintegrant. A lubricant will be preferably added to the blend to facilitate tablet formation.

It has been surprisingly found that the coated composition of the present invention exhibits superior chemical and physical stability as compared to composition containing polyvinyl alcohol in all the coating coatings or traditional tablets manufactured using conventional dry granulation or wet granulation.

The composition of the present invention may be formulated in suitable a dosage form including, but not limited to, a tablet, caplet, mini-tablet, pellets, granules, capsule filled with mini-tablets or pellets or combinations thereof.

The present invention further provides a method of treating type-II diabetes mellitus in a patient which method comprising administering the pharmaceutical composition as substantially described herein to the patient.

The present invention is further illustrated by the following examples which are provided merely to be exemplary of the invention and do not limit the scope of the invention. Certain modifications and equivalents will be apparent to those skilled in the art and are intended to be included within the scope of the present invention.

In accordance with the present Invention coated tablet formulations are set out below.

### Example 1

### Saxagliptin Tablet

TABLE 1

| | | |
|---|---|---|
| Sr. No. | Ingredients | Qty/Tab |

Core Tablets

| | | |
|---|---|---|
| 1 | Lactose Monohydrate | 98.00 |
| 2 | Microcrystalline Cellulose | 90.0 |
| 3 | Croscarmellose sodium | 10.0 |
| 4 | Magnesium Stearate | 2.00 |

| | |
|---|---|
| Total | 200.00 |

Drug Coating

| | | |
|---|---|---|
| 5 | Saxagliptin HCl anhydrous | 5.60 |
| | (Amorphus) | |
| 6 | Hypromellose or HPC or PVP or | 5.60 |
| | vinylpyrrolidone-vinyl acetate and | |
| | Glyceryl Caprylocaprate | |
| 7 | PEG 6000 | 1.00 |
| 8 | Talc | 1.00 |
| 9 | Titanium dioxide | 2.00 |
| 10 | Dichloromethane | qs |
| 11 | Methanol | qs |

Film Coating

| | | |
|---|---|---|
| 12 | Opadry | 5.0 |

| | |
|---|---|
| Total | 220.2 |

### Process:

Core tablet was prepared by mixing lactose, microcrystalline cellulose and croscarmellose sodium, followed by lubrication by mixing with magnesium stearate and compression.

A coating suspension was prepared by blending saxagliptin hydrochloride anhydrous with hypromellose/hydroxy propyl cellulose/PVP/vinylpyrrolidone-vinyl acetate and Glyceryl Caprylocaprate, PEG, Talc and Titanium dioxide.

The coating suspension was then coated over the core tablet and desired weight gain was achieved.

A film coat of opadry was then applied over the drug-coated tablet followed by drying of the film coat.

### Example 2

### Saxagliptin Tablet

TABLE 1

| Sr. No. | Ingredients | Qty in mg/Tablet |
|---|---|---|
| | Mixing and Blending | 2.5 mg |
| 1 | Lactose anhydrous | 98.00 |
| 2 | Microcrystalline cellulose | 90.00 |
| 3 | Cros carmellose sodium | 10.00 |

Lubrication

| | | |
|---|---|---|
| 4 | Magnesium stearate | 2.00 |
| | Target core tablet wt. | 200.00 |

Seal Coating

| | | |
|---|---|---|
| 5 | Hydroxypropyl Methylcellulose | 4.17 |
| 6 | Polyethylene glycol (PEG 4000) | 0.83 |
| 7 | Purified water | q.s |
| | ~ % Wt build up | 2.5 |
| | % w/w Solid contents | 5.0 |
| | Seal Coated Tablet wt | 205.00 |

Drug Coating

| | | |
|---|---|---|
| 8 | Saxagliptin Hydrochloride | 2.79 |
| 9 | Opadry II white 85F 18378 | 10.21 |
| 10 | Conc. HCl | q.s |
| 11 | Purified water | q.s |
| | ~ % Wt build up | 6.34 |
| | % w/w Solid contents | 8.0 |
| | Drug Coated Tablet wt | 218.0 |

Protective coating

| | | |
|---|---|---|
| 12 | Opadry II 85F540109 Pink | — |
| 13 | Opadry II 85F520082 Yellow | 5.00 |
| 14 | Conc. HCl | qs |
| 15 | Purified water | q.s |
| | ~ % Wt build up | 2.29 |
| | % w/w Solid contents | 15.0 |
| | Tablet wt | 223.00 |

### Process:

Lactose anhydrous, microcrystalline cellulose and croscarmellose sodium were blended. The blend was lubricated by blending with magnesium stearate. The lubricated blend was then compressed into tablets.

Hydroxypropyl methylcellulose was dissolve in purified water under constant vortex to form uniform solution. The compressed tablets were then coated using polymer solution with 65-70° C. of inlet temperature in coating pan.

Concentrated HCl was mixed in purified water to form 0.01N Hydrochloric acid. Saxagliptin HCl was dissolved in 0.01 N Hydrochloric acid under constant stirring to form clear solution. Opadry II white 85F 18378 was then added to the drug solution under constant stirring to form uniform dispersion. The above polymer coated tablets then coated with the drug and polymer solution with inlet temperature of 65-70° C. in coating pan.

Concentrated HCl was mixed in purified water to form 0.01 N Hydrochloric acid. Opadry II 85F540109 Pink/Opadry II 85F520082 Yellow was dispersed in half the quantity of 0.01 N Hydrochloric acid under constant stirring to form protective coating suspension. The above drug coated tablets then coated with protective coating suspension with inlet temperature of 65-70° C. in coating pan.

### Example 3

### Stability Study

The composition in accordance with the invention was subjected to stability study in two different packaging.

(I) Pack: 60 cc HDPE Thick Wall with 2 gm 2 in 1 Canister and 1 gm Absorbent Cotton

TABLE 3

| | | | 40° ± 2° C./75% ± 5 RH | | |
|---|---|---|---|---|---|
| Test | Details | Initial | 1M | 2M | 3M |
| 1 | Assay | 100.4 | 99.7 | 95.5 | 100.0 |
| 2 | Related Substance (%) | | | | |
| | Cyclic amide impurity | 0.000 | 0.056 | 0.251 | 0.499 |
| | Highest unknown | 0.031 | 0.052 | 0.043 | 0.056 |
| | Total Unknown | 0.056 | 0.261 | 0.210 | 0.301 |
| | Total Impurities | 0.056 | 0.317 | 0.461 | 0.823 |
| 3 | Dissolution test (0.1N HCl, 900 ml, USP-2, 50 RPM) | | | | |
| | Time points (min) | | | | |
| | 5 | 93 | 87 | 89 | 89 |
| | 10 | 100 | 99 | 100 | 95 |
| | 15 | 102 | 99 | 101 | 97 |
| | 20 | 102 | 100 | 102 | 98 |
| | 30 | 102 | 100 | 102 | 98 |

### (II) Alu-Alu Blister Pack

TABLE 3

| | | | 40° ± 2° C./75% ± 5 RH | |
|---|---|---|---|---|
| Test | Details | Initial | 1M | 3M |
| 1 | Assay | 100.4 | 95.4 | 99.7 |
| 2 | Related Substance (%) | | | |
| | Cyclic amide impurity | 0.000 | 0.075 | 0.585 |
| | Highest unknown | 0.031 | 0.049 | 0.058 |
| | Total Unknown | 0.056 | 0.188 | 0.291 |
| | Total Impurities | 0.056 | 0.263 | 0.897 |
| 3 | Dissolution test (0.1N HCl, 900 ml, USP-2, 50 RPM) | | | |
| | Time points (min) | | | |
| | 5 | 93 | 83 | 84 |
| | 10 | 100 | 92 | 96 |
| | 15 | 102 | 94 | 97 |
| | 20 | 102 | 94 | 98 |
| | 30 | 102 | 95 | 98 |

Result of the stability study indicates that saxagliptin composition in accordance with the present invention exhibits excellent storage stability.

1. A stable pharmaceutical composition of saxagliptin or salts, hydrates thereof comprising:
(a) at least one core;
(b) at least one first coating layer coated over the core comprising one or more pharmaceutical excipients, and optionally one or more polymers;
(c) at least one second coating layer disposed over the first coating layer comprising saxagliptin or salts, hydrated thereof, one or more pharmaceutical excipients, and optionally, one or more polymers; and
(d) optionally, an outer coating layer disposed over the second coating layer comprising one or more pharmaceutical excipients, and optionally, one or more polymers,
wherein said core does not contain saxagliptin, or salts, hydrates thereof and the composition is free of polyvinyl alcohol.

2. A stable pharmaceutical composition of saxagliptin or salts, hydrates thereof comprising:
(a) at least one core;
(b) at least one first coating layer coated over the core comprising one or more pharmaceutical excipients, and optionally one or more polymers;
(c) at least one second coating layer disposed over the first coating layer comprising saxagliptin or salts, hydrated thereof and one or more polymers; and
(d) optionally, an outer coating layer disposed over the second coating layer comprising one or more polymers,
wherein said core does not contain saxagliptin, or salts, hydrates thereof and said first coating layer is free of polyvinyl alcohol.

3. The stable pharmaceutical composition of claim 2, wherein the polymer in each of the second coating layer and the outer coating layer comprises polyvinyl alcohol.

4. The stable pharmaceutical composition of claim 1 or 2, wherein the first coating layer comprises one or more water-soluble polymer.

5. The stable pharmaceutical composition of claim 4, wherein the water-soluble polymer comprises hydroxypropyl methylcellulose.

6. The stable pharmaceutical composition of claim 1 or 2, wherein the first coating layer comprises from about 20% to about 99% w/w of polymer.

7. The stable pharmaceutical composition of claim 1 or 2, wherein the polymer in the first coating layer is different than the polymer in the second and outer coating layers.

8. The stable pharmaceutical composition of claim 1 or 2, wherein the composition retains at least 90% w/w of the total potency of saxagliptin after storage at 30° C. and 60% relative humidity for at least 3 months.

9. A process for preparation of stable pharmaceutical composition of claim 1 or 2, which process comprises of:
(a) providing at least one core;
(b) coating the core with at least one first coating layer comprising one or more pharmaceutical excipients, and optionally, one or more polymers;
(c) coating at least one second coating layer comprising saxagliptin or salts, hydrated thereof, one or more pharmaceutical excipients, and optionally, one or more polymers over the first coating layer;
(d) optionally, coating an outer coating layer comprising one or more pharmaceutical excipients, and optionally, one or more polymers over the second coating layer; and
(e) optionally, coating the outermost protective coating layer comprising one or more pharmaceutical excipients, colorant, and optionally one or more polymers over the outer coating layer.

10. A stable pharmaceutical composition of saxagliptin or salts, hydrates thereof comprising:
(a) at least one core;
(b) at least one first coating layer coated over the core comprising one or more polymers;
(c) at least one second coating layer disposed over the first coating layer comprising saxagliptin or salts, hydrated thereof and one or more polymers; and
(d) optionally, an outer coating layer disposed over the second coating layer comprising one or more polymers,
wherein said core does not contain saxagliptin, or salts, hydrates thereof and the first coating layer or the composition is free of polyvinyl alcohol.
